(19) United States
(12) Patent Application Publication
(10) Pub. No.: US 2018/0312899 A1
(43) Pub. Date: Nov. 1, 2018

(54) SYSTEM AND METHOD FOR RAPID MICROBIAL DETECTION AND ANALYSIS

(71) Applicant: Microban Products Company, Huntersville, NC (US)

(72) Inventors: Gina Parise Sloan, Statesville, NC (US); Jesse Douglas Turmenne, Mount Holly, NC (US); Joseph Ian Traynham, Huntersville, NC (US); Glenner Marie Richards, Davidson, NC (US); Michael Lynn Olson, China Grove, NC (US); Ivan Weikang Ong, Charlotte, NC (US)

(21) Appl. No.: 15/961,455

(22) Filed: Apr. 24, 2018

Related U.S. Application Data
(60) Provisional application No. 62/490,188, filed on Apr. 26, 2017.

(51) Int. Cl. C12Q 1/24; B01L 3/00; C12Q 1/06; G01N 1/40; G01N 27/74; G01N 21/29; G01N 27/00

(52) U.S. Cl. CPC C12Q 1/24; B01L 3/50825; C12Q 1/06; G01N 2001/002; G01N 27/74; G01N 21/29; G01N 27/00; G01N 1/40

(57) ABSTRACT

A method and system for rapid microbial detection and analysis comprising a sampling device, a swab with a swab head connected to a swab handle having analysis fluid therein, a collection chamber, and a detection and analysis device

### CROSS-REFERENCE TO RELATED APPLICATION

This application claims priority from U.S. provisional patent application No. 62/490,188, filed on Apr. 26, 2017, in the United States Patent and Trademark Office. The disclosure of which is incorporated herein by reference in its entirety.

### FIELD OF THE INVENTION

The present invention generally relates to microbial detection and analysis, more particularly to a system and method for rapid microbial detection and analysis.

### BACKGROUND OF THE INVENTION

Rapid microbial detection is a need for multiple industries attempting to reduce the risk of cross contamination or the spread of illness. The term “rapid” typically refers to detection within a matter of seconds to minutes as opposed to hours. Current technologies can be classified into two groups. They are either low cost, low accuracy, with results within minutes (i.e. rapid) or they are high cost, high accuracy, with results within hours. The low cost options are not able to accurately identify the true problem, viable microbial bioburden, as opposed to dust, dirt, grime, or other non-viable presence on the surface. Viable microbial bioburden is important to accurately understand potential health risks or conduct microbiological experiments. The low cost options include the use of ATP luminescence technology or fluorescent markers. Both of these options provide feedback to the user within seconds to minutes. High cost options are typically utilized to diagnose patients or release lots for distribution following food manufacture. These high cost systems act within hours, require sampling, sample transit and trained personnel to run the test.

Thus, there is a growing industry need for a low cost, highly accurate and easy to use system that can detect the presence of viable microorganisms. The system and method of the present invention are designed to overcome the above disadvantages and meet this industry need.

### SUMMARY OF THE INVENTION

Microbial sampling is typically conducted in order to provide a manner of removing and analyzing microbial samples from multiple substrates and matrices. The system and method of the present invention allows for the low cost, accurate and quick identification of microbial contamination and presence.

The system of the present invention for microbial detection and analysis comprises a sampling device that can remove bacteria from a surface, measure or tabulate the surface area of the surface that has been sampled and, when appropriate for analysis, rapidly remove the bacteria into a specific analysis solution.

The system comprises a collection chamber that acts through capillary action or via a microfluidic collection device to enable rapid sampling of liquids.

The system comprises a detection device utilizing a colorimetric indication or magnetic levitation or an alternate technique, such as acoustic waves, within a chamber(s) to result in viable cell recognition within minutes and an imaging device to image the samples, via holography or microscopy.

Further areas of applicability of the present invention will become apparent from the detailed description provided hereinafter. It should be understood that the detailed description and specific examples, while indicating the preferred embodiments of the invention, are intended for purposes of illustration only and are not intended to limit the scope of the invention.

### BRIEF DESCRIPTION OF THE DRAWINGS

The present invention will become more fully understood from the detailed description and the accompanying drawings, which are not necessarily to scale, wherein:

FIG. 1 is an illustration of a system for microbial detection and analysis in accordance with the present invention.

FIG. 2 is an illustration of a sampling device of the microbial detection system of FIG. 1.

FIG. 3 is an illustration of an entry port of the collection chamber of FIG. 1.

FIGS. 4A and 4B are illustrations of the collection chamber shown in FIG. 1.

FIG. 5 illustrates fluid flow into one collection port of the collection chamber shown in FIG. 1.

FIG. 6 illustrates fluid flow into a microfluidic device with multiple channels.

FIG. 7 is an illustration of the detection and analysis device shown in FIG. 1.

FIG. 8 is an illustration of a method in accordance with the present invention.

FIGS. 9 and 10 are illustrations of the swab handle of the sampling device.

FIGS. 11 and 12 are illustrations of a physical barrier in a form of a ring for the sampling device.

### DESCRIPTION OF THE PREFERRED EMBODIMENTS

The following description of the embodiments of the present invention is merely exemplary in nature and is in no way intended to limit the invention, its application, or uses. The present invention has broad potential application and utility, which is contemplated to be adaptable across a wide range of industries. The following description is provided herein solely by way of example for purposes of providing an enabling disclosure of the invention, but does not limit the scope or substance of the invention.

As used herein, the terms “microbe” or “microbial” should be interpreted to refer to any of the microscopic organisms studied by microbiologists or found in the use environment of a treated article. Such organisms include, but are not limited to, bacteria and fungi as well as other single-celled organisms such as mold, mildew and algae. Viral particles and other infectious agents are also included in the term microbe.

The term “substrate(s)” or “matrices” is understood to include both animate and inanimate surfaces. Such animate surfaces may include, but are not limited to, skin, mucosal layers, muscular tissue that are of human or animal origin. Inanimate surfaces include, but are not limited to, any surface that is non-biological in nature.

The term “gear” as used in this disclosure and the appended claims refers to any device useful for continuous measurement of a motion path. The term “gear” includes, but is not limited to, a traditional toothed gear. Other examples of such devices include, but are not limited to, a stepper, a motor, and an electrical transducer.

Further, the term “or” as used in this disclosure and the appended claims is intended to mean an inclusive “or” rather than an exclusive “or.” That is, unless specified otherwise, or clear from the context, the phrase “X employs A or B” is intended to mean any of the natural inclusive permutations. That is, the phrase “X employs A or B” is satisfied by any of the following instances: X employs A; X employs B; or X employs both A and B. In addition, the articles “a” and “an” as used in this application and the appended claims should generally be construed to mean “one or more” unless specified otherwise or clear from the context to be directed to a singular form. Throughout the specification and claims, the following terms take at least the meanings explicitly associated herein, unless the context dictates otherwise. The meanings identified below do not necessarily limit the terms, but merely provided illustrative examples for the terms. The meaning of “a,” “an,” and “the” may include plural references, and the meaning of “in” may include “in” and “on.” The phrase “in one embodiment,” as used herein does not necessarily refer to the same embodiment, although it may.

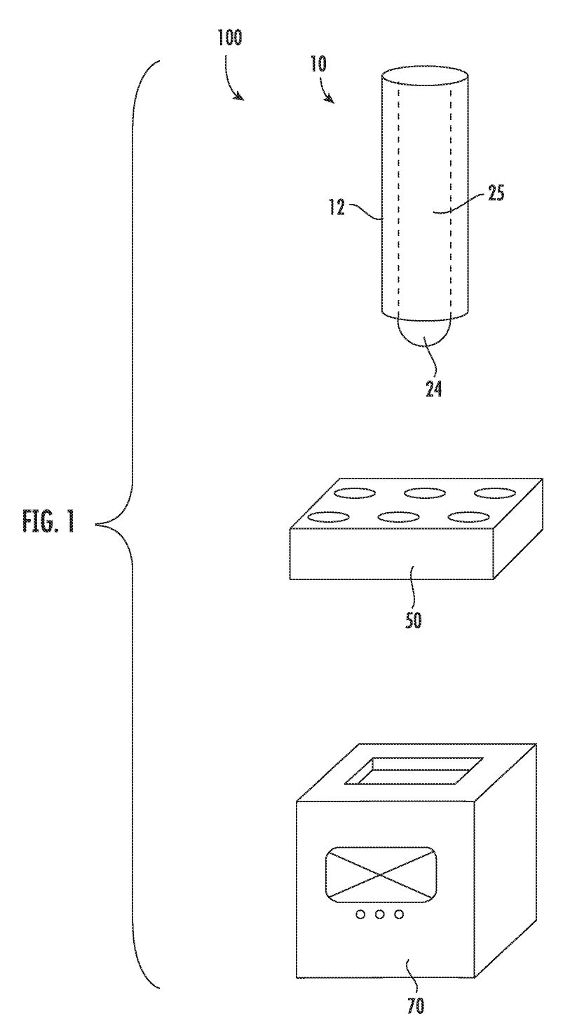

FIG. 1 is an illustration of a system 100 for microbial analysis and detection in accordance with the present invention. As shown in FIG. 1, system 100 generally comprises a sampling device 10, a collection chamber 50, and a detection and analysis device 70. Sampling device 10 is in fluid communication with each of collection chamber 50 and detection and analysis device 70. The fluid in collection chamber 50 is visualized/imaged in detection and analysis device 70.

### Sampling Device

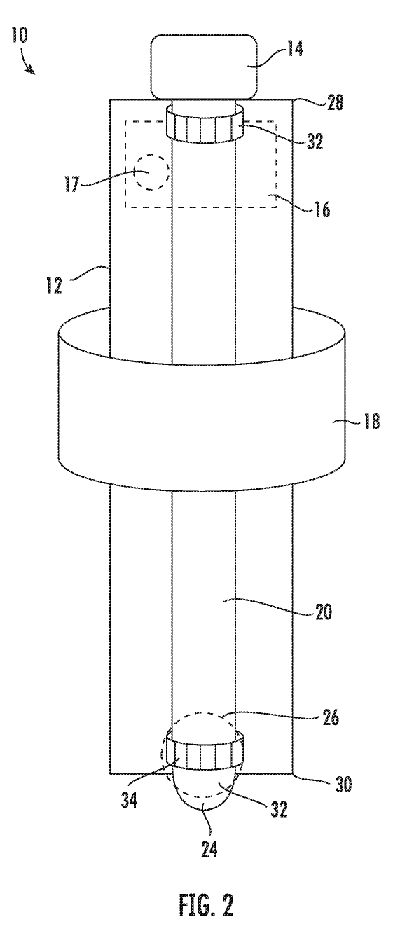

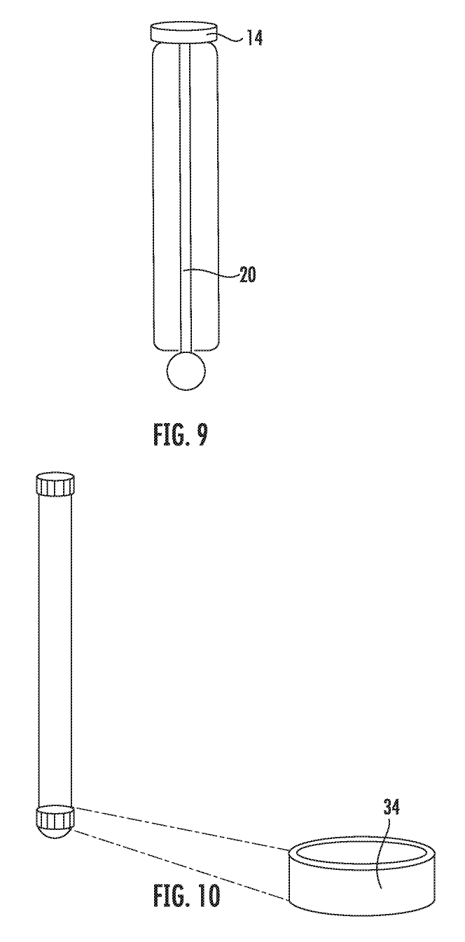

Referring to FIGS. 1 and 2, sampling device 10 comprises a housing 12, an ejection mechanism (such as a button) 14 attached to or connected to housing 12, a counter mechanism 16 housed within housing 12, a sampling device gear 17 housed within housing 12, and a physical barrier 18 (such as in a form of a ring) surrounding housing 12 and movable in the vertical direction of sampling device 10. A swab 25, having a swab handle 20 and a swab head 24, is disposable. Swab 25 is insertable within housing 12 and ejectable from housing 12 by ejection mechanism 14, also shown in FIG. 9. A connector 26 connects swab handle 20 to swab head 24. Preferably, connector 26 is internal to swab handle 20. Swab head 24 is the portion of sampling device 10 that contacts and interacts with a surface having bacteria/microbes thereon.

Swab handle 20 has a first end 28 and a second end 30. It is contemplated and within the scope of the present invention that swab handle 20 has at least one gear. As illustrated in FIG. 2, swab handle 20 comprises a first gear 32 near first (top) end 28 of swab handle 20 and a second gear 34 near second (bottom) end 30 of swab handle 20. Second gear 34 is also shown in FIG. 10.

Swab handle 20 is hollow allowing for an analysis fluid (not shown) to be trapped or contained inside swab handle 20. Connector 26 is preferably internal to swab handle 20. Connector 26 is, for example, a one way-valve, a polymer seal, or an alternate sealant that connects swab handle 20 to swab head 24. Connector 26 is used to prevent analysis fluid from within swab handle 20 from traveling to swab head 24 before a user is ready to engage the system. Swab head 24 is located near second end 30 of swab handle 20.

Housing 12 of sampling device 10 comprises sampling device gear 17. Sampling device gear 17 rotates when engaged with first gear 32 of swab handle 20 to tabulate a target area (such as in cm²) being sampled. Once the target area has been reached, sampling device 10 alerts or notifies a user that sampling is complete.

Sampling device gear 17 in sampling device 10 counts down from the target surface area. Sampling device gear 17 is calibrated such that each rotation corresponds to a known area traveled. Sampling device gear 17 is engaged once swab head 24 engages with sampling device 10. The engagement of swab head 24 with sampling device 10 can occur by various mechanisms including, but not limited to, a Leur lock connection, a snap on connection utilizing a roller ball in socket type of joint, a sleeve fit, among others.

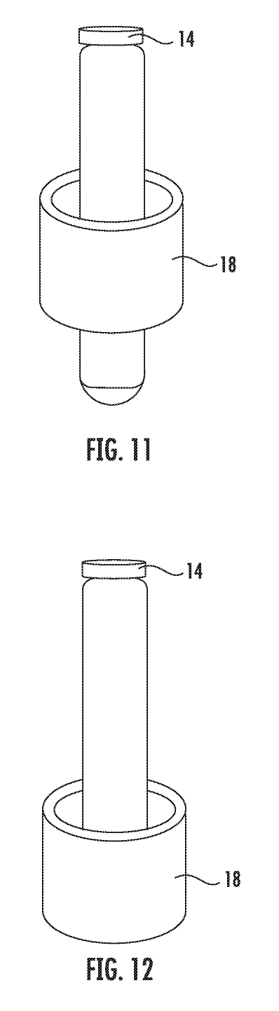

Swab head 24 rotates by engaging a swivel wheel or ball mechanism 32 (either internal to swab head 24 as shown in FIG. 2 or external thereto). As swab head 24 rotates, the rotation of the roller ball or swivel wheel or other mechanism 32 that either is affixed to swab head 24 or is engineered within swab head 24 engages and turns second gear 34. As second gear 34 turns, the swab handle 20 rotates and first gear 32 engages sampling device gear 17. As calibrated sampling device gear 17 turns, the internal counter 16 decreases until the target set sample area has been reached. Once the set sample area has been reached, the user is notified by a notification mechanism including, but not limited to, an electronic signal resulting in a light or sound, a physical mechanism, and a combination thereof. With an electronic signal resulting in a light or sound, the electronic signal is engaged once the counter 16 has reached the end point, and this aligns conductors that finalize a circuit within sampling device 10. The completion of the circuit engages a LED light, a speaker, or a combination thereof. With regard to barrier 18, once counter 16 reaches the end point, counter 16 engages with an accentuator (not shown) that is behind barrier 18 and that holds the barrier 18 in place. Barrier 18 is engaged with sampling device 10 via elastomer compression, compression spring, or helical escapement mechanism via a coiled incline plane. Thus, the release of the accentuator causes release of the springs, etc. and thus forces barrier 18 down. FIGS. 11 and 12 are illustrations of physical barrier 18 in a form of a ring for the sampling device. Once counter mechanism 16 reaches the end point, barrier 18 is released and drops down to cover swab head 24 and to cease sampling. Barrier 18 may lift swab head 24 slightly off the surface, causing cessation of sampling. Once a new swab is inserted the barrier is reset in conjunction with the counter mechanism. Barrier 18 is preferably constructed of plastic, metal, or a combination thereof.

Swab head 24 is designed so as to easily remove bacteria once the analysis fluid, from swab handle 20, has been introduced to swab head 24. This can be accomplished via a number of methods unique to the invention.

A method for removal of bacteria from swab head 24 is use of a thermopolymeric fiber for swab head 24 that only dissolves when introduced into the analysis fluid at a temperature that matches the thermodegradation profile of the polymer of the thermopolymeric fiber.

Another method for removal of the bacteria from swab head 24 is utilization of a biopolymer based fiber that is placed in an analysis fluid that is optimally formulated for the activity of an enzymatic digestion of the biopolymer. Non-limiting examples of a biopolymer based fiber include, but are not limited to: (a) poly-N-acetyl glucosamine as the polysaccharide based fiber paired with analysis fluid that contains Dispersin B; (b) cellulose based polysaccharide fiber that is paired with a cellulase; (c) protein fiber that is paired with an appropriate proteinase or mix of proteinases to cause rapid dissolution of the fiber, where non-limiting examples of enzymes to pair with protein fiber materials include, but are not limited to, proteinase K, trypsin, chymotrypsin, etc.; (d) nucleic acid based fiber that is paired with either DNase or RNase depending on the origin of the fiber being either ribonucleic acid or deoxyribonucleic acid.

Another method for removal of bacteria from swab head 24 is utilization of magnetic beads that are located within the analysis fluid. Once the analysis solution passes over swab head 24, the magnetic beads within the analysis fluid bind to the organic debris. Magnetic beads can be coated with molecules that have an affinity for the microbial cell wall. Magnetic beads are commercially available and sources are known by those of ordinary skill in the art. After swab head 24 has been saturated with the analysis fluid, the analysis fluid is introduced into a weak magnetic or electrical field within the collection chamber such that the magnetic particles (now attached to the organic material) are removed from swab head 24 into the analysis fluid.

In yet another method, swab head 24 is comprised of a material with a defined pore size that is able to generate sufficient fluidic pressure to cause the release of microbes from swab head 24. The fluidic pressure is generated once the seal formed by connector 26 between swab handle 20 and swab head 24 is broken. The analysis fluid is forced through pores of swab head 24 via the depression of a plunger or sterile air into the hollow space within swab handle 20. The small size (preferably on the order of sub-micrometers) of the pores causes turbulence and pressure within the fluid that dislodges any adherent cells into the fluid as it passes over swab head 24. The pressure exerted upon swab head 24 forces the liquid out of swab head 24 and into collection chamber 50.

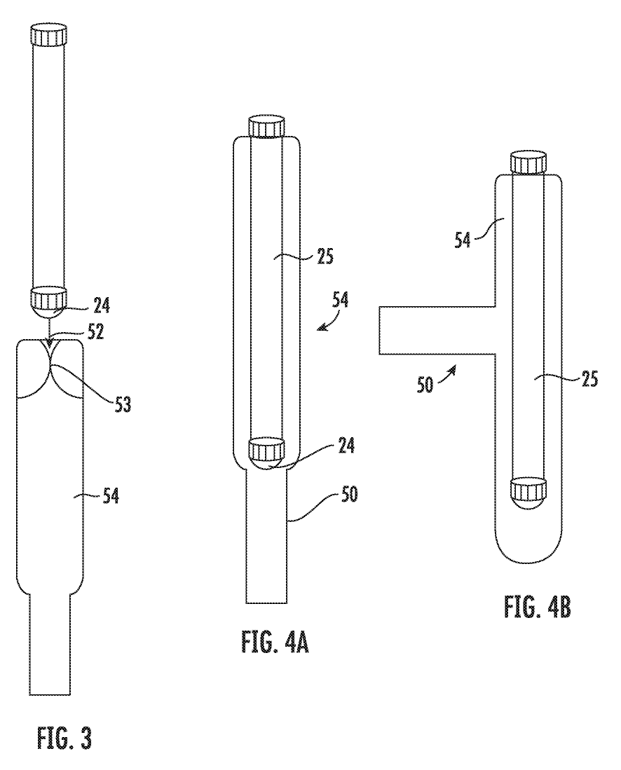

In still yet another method, swab head 24 is placed into an entry port 52 of a cap 54, as shown in FIG. 3. Entry port 52 is comprised of a flexible polymeric material with an orifice 53 smaller than swab head 24. Pressure exerted upon swab head 24 as it passes through orifice 53 forces the liquid/analysis fluid out of swab head 24 and into collection chamber 50.

### Collection Chamber

Referring to FIGS. 4A and 4B, swab 25 is introduced into a cap 54 for the liquid/fluid expressed from swab head 24 (such as by one of the above methods) to be collected in collection chamber 50. Cap 54 is a cover for swab 25 including swab head 24. Collection chamber 50 can have one or more collection areas or ports. Collection chamber 50 is part of cap 54. When collection chamber 50 is at the bottom of cap 54, as shown in FIG. 4A, the cap is filled via gravity and capillary action. Collection chamber 50 can also be located at different positions along cap 54 as shown in FIG. 4B. If at a different location, cap 54 would need to be turned to facilitate fill of collection chamber 50.

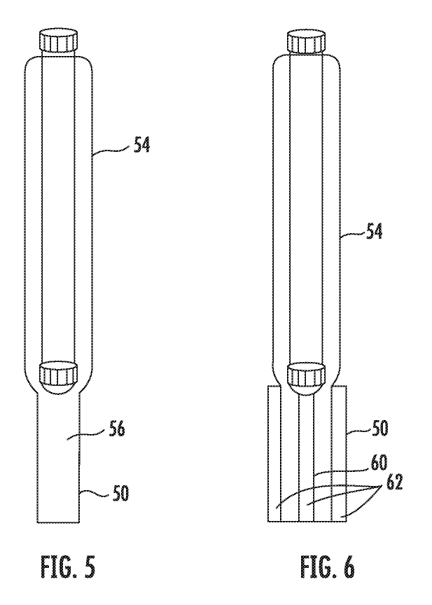

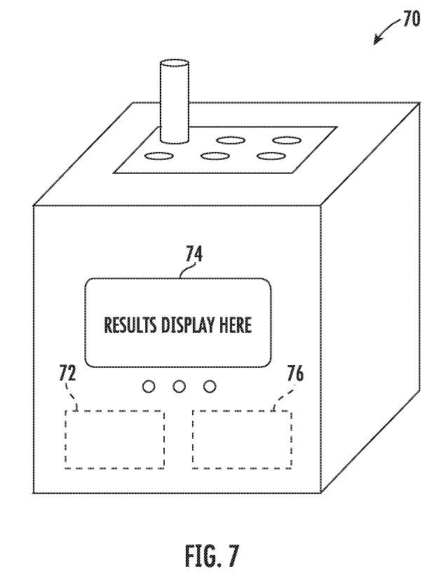

Referring to FIG. 5, there is one collection channel or port 56 within collection chamber 50 for sampling the fluid, and fluid flows into one port 56. In FIG. 6, collection chamber 50 has a microfluidic device 60 that is able to make multiple samples at one time. Fluid flows into microfluidic device 60 with multiple channels or ports 62 to collect the fluid. Collection chamber 50 is in fluid communication with the liquid/fluid received from swab head 24. Collection chamber 50 is constructed of a metal, a polymer, or other material(s), is optically clear, and optionally has a coating applied thereon to facilitate fluid flow.

Thus, the collection port may have a configuration of a simple glass square capillary in which the capillary is optionally covered with an optically clear polymer sheath to prevent breakage, or the collection port may have a configuration of a microfluidic device made of an optically clear polymer, such as but not limited to, polycarbonate. The microfluidic device samples the same volume of the analysis solution across 5 to 10 capillaries. The increased sampling ports allow for increased resolution and sensitivity of the assay.

### Detection and Analysis Device

Referring to the figures, system 100 comprises a detection and analysis device 70 for insertion of collection chamber 50 into detection and analysis device 70. Detection and analysis device 70 comprises a magnetic or electrical field 72.

In accordance with the method of the invention, a user inserts a portion of collection chamber 70 that contains the capillary(s) into detection and analysis device 70. In a preferred embodiment, detection and analysis device 70 holds one or more (for example, up to 50) capillaries depending upon the incorporation of an auto-sampling port. The capillaries, once inserted, enter magnetic or electrical field 72 that is uniform across all samples. Alternatively, field 72 can incorporate an electronic field including, but not limited to, a piezoelectric field, a DC/AC field, or a combination thereof. The user initiates a timer once all samples are inserted that allows samples to sit for the required analysis time. The required analysis time can span from seconds to 20 minutes, for example. Once the time is complete, a belt (not shown) moves the samples into an analysis port 76 within detection and analysis device 70. If there is only one sample, detection and analysis device 70 may be hand-held, for example. If there is only one sample, detection and analysis device 70 does not need to contain a belt such that the sample sits within the magnetic field or electrical field and is imaged at the same location. The samples are imaged using either holography, traditional microscopy techniques that are known to those skilled in the art, or a combination thereof. The images are analyzed. Preferably, the images are analyzed using an algorithm primarily for counting pixels and corresponding pixels to a cell count. Non-limiting examples of such algorithms are algorithms for particle counters based on white and black pixels (see, for example, http://imagej.net/Particle_Analysis#Automatic_Particle_counting). Alternatively, in lieu of photographs, the viable bacteria can be counted as they pass over a sensor. The sensor may be an occlusion sensor such as IR or light sensor. The sensor can also utilize dynamic light scattering or any other form of light detection for counting viable microorganism.

A report and/or graph is computer generated from a computer housed within detection and analysis device 70 or in communication with detection and analysis device 70. The report differentiates viable vs. non-viable bacteria. The user is notified of the number of viable bacteria by any number of possible methods.

In one such method, if the number of viable bacteria is higher than a user set, pre-determined threshold, a display screen 74 provides a visual indicator such as the color red or another color, for example, indicating the area needs to be cleaned. If the number of viable bacteria is less than the user set, pre-determined threshold, display screen 74 shows another visual indicator such as shows the color green or another color, for example, indicating that the sampled area is not of concern.

In another method, display screen 74 shows the user the number of viable microbes per sampled surface area.

A combination of the above methods can be used in which the user receives a red or green notification and the true number of bacteria is transmitted to a database that can be accessed by authorized personnel.

The above is a non-exhaustive list of methods and any number of methods of notification may be used and are contemplated to be within the scope of the present invention.

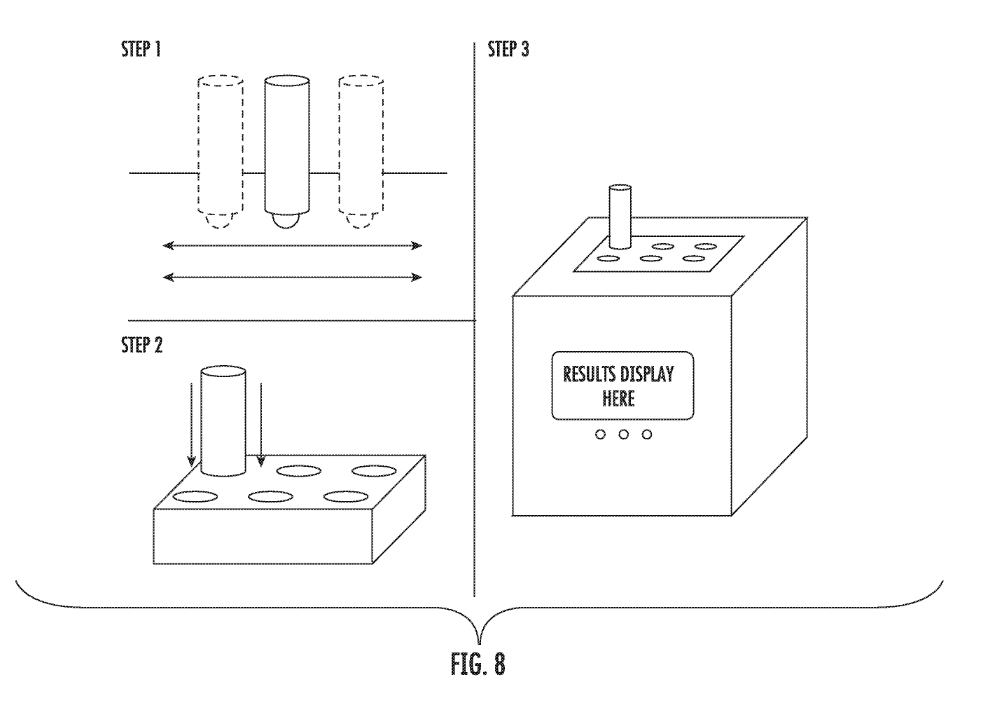

FIG. 8 is an illustration of a method in accordance with the present invention. As shown in FIG. 8, the method generally comprises sampling bacteria from a surface with a sampling device having a swab head connected to a swab handle having analysis fluid therein, wherein the swab head is in contact with the surface having bacteria thereon (step 1); introducing the analysis fluid from the swab handle of the sampling device to the swab head and removing bacteria from swab head with the analysis fluid to collect the analysis fluid and bacteria in a collection chamber (step 2); inserting the collection chamber with analysis fluid and bacteria in the detection and analysis device, wherein the detection and analysis device has a magnetic field, electrical field, or electronic field, and reader chamber for computer generating a report of viable bacteria (step 3).

It will therefore be readily understood by those persons skilled in the art that the present invention is susceptible of broad utility and application. Many embodiments and adaptations of the present invention other than those herein described, as well as many variations, modifications and equivalent arrangements, will be apparent from or reasonably suggested by the present invention and the foregoing description thereof, without departing from the substance or scope of the present invention. Accordingly, while the present invention has been described herein in detail in relation to its preferred embodiment, it is to be understood that this disclosure is only illustrative and exemplary of the present invention and is made merely for purposes of providing a full and enabling disclosure of the invention. The foregoing disclosure is not intended or to be construed to limit the present invention or otherwise to exclude any such other embodiments, adaptations, variations, modifications and equivalent arrangements.

What is claimed is:

1. A system for rapid microbial detection and analysis comprising:
a sampling device,
a swab having a swab head connected to a swab handle having analysis fluid therein for insertion in the sampling device,
a collection chamber, and
a detection and analysis device,
wherein the sampling device is in fluid communication with the collection chamber and the detection and analysis device.

2. The system according to claim 1, wherein the sampling device comprises a housing and an ejection mechanism attached to or connected to the housing.

3. The system according to claim 2, further comprising a counter mechanism housed within the housing, a sampling device gear housed within the housing, and a physical barrier surrounding the housing and movable in a vertical direction of the sampling device.

4. The system according to claim 3, wherein the physical barrier is in a form of a ring.

5. The system according to claim 1, wherein the swab is disposable.

6. The system according to claim 2, wherein the swab is insertable within the housing.

7. The system according to claim 2, wherein the swab is ejectable from the housing by the ejection mechanism.

8. The system according to claim 1, wherein a connector connects the swab handle to the swab head.

9. The system according to claim 8, wherein the connector is internal to the swab handle.

10. The system according to claim 8, wherein the connector is a one way-valve, a polymer seal, or an alternate sealant that connects the swab handle to the swab head.

11. The system according to claim 1, wherein the swab handle is hollow.

12. The system according to claim 1, wherein the swab handle contains an analysis fluid.

13. The system according to claim 3, wherein the swab handle comprises a first gear near a first end of the swab handle and a second gear near the second end of the swab handle.

14. The system according to claim 13, wherein the sampling device gear rotates when engaged with the first gear of the swab handle to tabulate a target area being sampled.

15. The system according to claim 3, wherein the sampling device gear is calibrated with each rotation corresponding to a known area traveled.

16. The system according to claim 3, wherein the sampling device gear is engaged upon engagement by the swab head with the sampling device.

17. The system according to claim 16, wherein the engagement of the swab head with the sampling device is by a mechanism selected from the group consisting of a Leur lock connection, a snap on connection, a roller ball in socket type of joint, a sleeve fit, and a combination thereof.

18. The system according to claim 1, further comprising a notification mechanism selected from the group consisting of an electronic signal resulting in a light or sound, a physical mechanism, and a combination thereof.

19. The system according to claim 3, wherein the counter mechanism engages with an accentuator behind the physical barrier and that holds the physical barrier in place.

20. The system according to claim 3, wherein the physical barrier is engaged with the sampling device by elastomer compression, a compression spring, a helical escapement mechanism via a coiled incline plane, and a combination thereof.

21. The system according to claim 1, wherein the swab head comprises a fiber selected from the group consisting of a thermopolymeric fiber, a biopolymer based fiber, and a combination thereof.

22. The system according to claim 21, wherein the biopolymer based fiber is selected from the group consisting of poly-N-acetyl glucosamine, a polysaccharide based fiber, a cellulose based polysaccharide fiber, a protein fiber, a nucleic acid based fiber, and a combination thereof.

23. The system according to claim 1, wherein magnetic beads are located within the analysis fluid.

24. The system according to claim 1, wherein the swab head is comprised of a material with a pore size on an order of sub-micrometers.

25. The system according to claim 1, wherein the collection chamber has at least one collection area or port.

26. The system according to claim 1, wherein the collection chamber is part of a cap.

27. The system according to claim 1, wherein the collection chamber comprises a microfluidic device.

28. The system according to claim 1, wherein the collection chamber is optically clear.

29. The system according to claim 1, wherein the collection chamber has a coating applied thereon.

30. The system according to claim 1, wherein the collection chamber is insertable into the detection and analysis device.

31. The system according to claim 1, wherein the detection and analysis device comprises a field selected from the group consisting of a magnetic field, an electrical field, an electronic field, and a combination thereof.

32. The system according to claim 1, wherein the detection and analysis device comprises an analysis port.

33. The system according to claim 1, wherein the detection and analysis device is hand held.

34. The system according to claim 1, wherein the detection and analysis device comprises a mechanism for imaging a sample.

35. The system according to claim 1, wherein the detection and analysis device comprises or has access to a pixel-counting algorithm.

36. The system according to claim 1, wherein the detection and analysis device comprises a sensor.

37. The system according to claim 1, wherein the detection and analysis device comprises a reader chamber for determining viable bacteria.

38. The system according to claim 1, wherein the detection and analysis device comprises a visual indicator of bacteria or lack thereof.

39. The system according to claim 1, wherein the detection and analysis device comprises a colorimetric indication, magnetic levitation, or both.

40. A method for rapid microbial detection and analysis comprising:
sampling bacteria from a surface with a sampling device having an inserted swab with a swab head connected to a swab handle having an analysis fluid therein, wherein the swab head is in contact with the surface having bacteria thereon;
introducing the analysis fluid from the swab handle of the sampling device to the swab head;
removing bacteria from swab head with the analysis fluid;
collecting the analysis fluid and bacteria in a collection chamber; and
inserting the collection chamber with analysis fluid and bacteria in the detection and analysis device, wherein the detection and analysis device has a magnetic field, electrical field, or electronic field, and a reader chamber for determining viable bacteria.
